(19) United States
(12) Patent Application Publication
(10) Pub. No.: US 2025/0276120 A1
(43) Pub. Date: Sep. 4, 2025

(54) METHOD FOR PREVENTING COAGULATION IN PATIENTS WITH IMPLANTED CARDIAC DEVICES

(71) Applicant: Cadrenal Therapeutics, Inc., Ponte Vedra, FL (US)

(72) Inventor: Quang PHAM, Ponte Vedra, FL (US)

(21) Appl. No.: 19/067,803

(22) Filed: Feb. 28, 2025

Related U.S. Application Data
(60) Provisional application No. 63/560,351, filed on Mar. 1, 2024.

(51) Int. Cl. A61M 1/36; A61F 2/24; A61M 60/178

(52) U.S. Cl. CPC A61M 1/3672; A61F 2/24; A61M 60/178

(57) ABSTRACT

A method of inhibiting coagulation in a patient having undergone implantation of a cardiac device includes administering tecarfarin to the patient, in an amount and for a period of time, effective to inhibit coagulation. The cardiac device could be a blood pump, prosthetic heart valve or a coronary stent.

### CROSS REFERENCE TO RELATED APPLICATIONS

This application claims priority to U.S. Provisional Application No. 63/560,351 filed on Mar. 1, 2024. The aforementioned application is incorporated by reference herein in its entirety.

### TECHNICAL FIELD

The present invention provides for a method for inhibiting coagulation in patients who have undergone implantation of a cardiac device. This method includes administering tecarfarin to patients in an amount sufficient to effectively and reliably inhibit coagulation. The cardiac device could be a blood pump, prosthetic heart valve or coronary stent.

### BACKGROUND OF THE INVENTION

Coagulation refers to the process by which blood or another biological fluid changes from a liquid to a gel or semi-solid state, leading to clot formation. A blood clot is a mass of blood that forms when blood platelets, fibrin, and cells aggregate within the vascular system. The blood clot can include a pathological clot formation in which blood coagulates abnormally within the veins or arteries, potentially leading to vascular blockages as well as risk of embolization, and pose significant health risks such as arterial occlusion, acute myocardial infarction, stroke, deep vein thrombosis and pulmonary embolism.

Vitamin K is required for the production of certain proteins involved in the blood clotting process. The Vitamin K-dependent coagulation factors (II, VII, IX, X, proteins C and S) require gamma carboxylation of glutamic acid residues that allow individual coagulation factors to bind to phospholipid surfaces on platelets and the vascular endothelium. The carboxylation reaction can proceed only if accompanied by the conversion of a reduced form of vitamin K (vitamin K hydroquinone) to vitamin K epoxide. Vitamin K epoxide is, in turn, recycled back to vitamin K and vitamin K hydroquinone by another enzyme, Vitamin K epoxide reductase.

Vitamin K Antagonists are a form of antithrombotic therapy that inhibit synthesis of vitamin K-dependent clotting factors. They are structurally similar to Vitamin K and deplete the active form of Vitamin K by inhibiting Vitamin K epoxide reductase and interfering with the recycling of inactive Vitamin K epoxide back to the active reduced form of vitamin K necessary for synthesis of Vitamin-K dependent clotting factors.

Warfarin is the most commonly used Vitamin K antagonist and has been in clinical use for more than 60 years. It is still widely used in clinical circumstances where chronic oral anticoagulation is necessary, especially in circumstances where monitoring can facilitate consistent degrees of anticoagulation. However, there are also some clinical circumstances where warfarin is less reliable, and it may be difficult to maintain patients within a relatively narrow range of therapeutic anticoagulation.

One important reason for this is that warfarin is a racemic mixture of (R)- and (S)-enantiomers, which are metabolized by multiple different isoenzymes of the cytochrome P450 system (primarily CYP2C9, CYP1A2 and CYP3A4) with resulting potential for numerous food and drug interactions. Additionally, other factors such as genetic polymorphisms (including CYP2C9 variants), age, concomitant diseases and disorders of hepatic and renal function may significantly affect drug levels and increase the likelihood of over- and under-dosing which, in turn, are associated with an enhanced risk of bleeding or thrombotic complications, respectively. More frequent excursions outside the relatively narrow individualized therapeutic range require additional dose adjustments, and greater difficulty in establishing a consistently reliable degree of anticoagulation. A narrow therapeutic range also necessitates more frequent monitoring of the International Normalized Ratio (INR) to maintain effective anticoagulation while avoiding bleeding risks. Studies have shown that a substantial percentage of patients on warfarin spend a considerable amount of time outside the target INR range, often as high as 40-60%, leading to increased risks of both thromboembolic and hemorrhagic events.

Furthermore, warfarin's effectiveness is greatly influenced by interactions with various medications and dietary factors, which can alter its metabolism and anticoagulant effects, necessitating constant adjustments in dosage. Genetic variations in enzymes involved in warfarin metabolism also contribute to unpredictable responses, requiring individualized dosing strategies and more frequent monitoring. Renal function also plays a significant role, given the excretion of warfarin metabolites by the kidney, and the well-recognized higher bleeding rates seen in dialysis patients. Hepatic function is important, both from the standpoint that the liver is the manufacturing site for many coagulation factors and a potential primary site for metabolizing certain drugs (like warfarin). All these factors combine to create a complex management landscape for warfarin, demanding significant patient education, adherence to monitoring schedules, and careful consideration of potential interactions to ensure optimal and safe anticoagulation therapy.

Given the challenges of warfarin, clinical consequences associated with these limitations are significant, particularly for patients with implanted cardiac devices, in whom precise control of coagulation is paramount. For example, patients with LVADs maintained on warfarin have annual stroke rates ranging from 2% to 10%, and major bleeding rates from 10% to as high as 30%, emphasizing the precarious balance required with warfarin anticoagulation in higher-risk populations.

While direct oral anticoagulants (DOACs) have emerged as alternatives to warfarin for chronic anticoagulation in the prophylaxis and treatment of DVT and in preventing thromboembolic events in the general population of non-valvular atrial fibrillation, their use in patients with implanted cardiac devices such as ventricular assist devices (VADs), prosthetic mechanical valves, and in chronically anticoagulated patients undergoing coronary stenting remains limited and controversial. The efficacy and safety of DOACs, including factor Xa inhibitors (rivaroxaban, apixaban, edoxaban) and direct thrombin inhibitors (dabigatran) in the context of implanted cardiac devices has not been well-established, primarily due to the lack of large-scale randomized controlled trials in these specific patient populations. Moreover, concerns exist regarding the inability to rapidly reverse the anticoagulant effects of DOACs in emergency situations, which is particularly crucial for higher risk cardiac patients who may require urgent surgical interventions or face life-threatening bleeding complications.

Furthermore, the pharmacokinetics and pharmacodynamics of DOACs may be altered in patients with severe heart failure due to changes in drug absorption, distribution, and elimination associated with the device and underlying cardiovascular pathology. Additional challenges arise due to the lack of a standardized monitoring method for DOACs in managing anticoagulation in these high-risk populations, where precise control of anticoagulation is critical, and some DOACs have even shown increased bleeding risks in patients with mechanical heart valves.

One group of patients in whom achieving reliable anticoagulation is particularly problematic is patients with left ventricular assist devices (LVADs), where the therapeutic window for avoiding the life-threatening complication of pump thrombosis without major bleeding risks is especially narrow, especially in the frequently encountered setting of liver or kidney impairment. The potential for clinical application in this area has been recognized by the FDA's orphan drug designation for tecarfarin in preventing thromboembolism and thrombosis in patients with implanted mechanical circulatory support devices.

Another group of patients where precise anticoagulation is essential is patients with prosthetic heart valves, who require anticoagulation therapy to prevent blood clots. Patients with mechanical heart valves require lifelong anticoagulation as these patients have a higher risk of stroke and valve thrombosis, and patients with bioprosthetic valves also need anticoagulation for the first three to six months after surgery.

A third group of patients where chronic anticoagulation is challenging is patients who are undergoing a percutaneous coronary revascularization with a stent who are already on oral anticoagulants for another reason, and who require the addition of potent dual antiplatelet therapy to their existing anticoagulation regimen.

Finally, as noted, metabolic derangements associated with impaired kidney and liver function (which can frequently be present in critically ill patients) further complicate effective chronic anticoagulation.

Overall, there is a significant unmet need for a more reliable and predictable anticoagulant with fewer interactions and a wider therapeutic window for patients with implanted cardiac devices who require chronic antithrombotic therapy.

### SUMMARY OF THE INVENTION

In one aspect of the present invention a method of inhibiting coagulation in a patient having undergone implantation of a cardiac device includes administering tecarfarin to the patient, in an amount and for a period of time, effective to inhibit coagulation. The cardiac device could be a blood pump, a prosthetic heart valve or a coronary stent.

### BRIEF DESCRIPTION OF THE FIGURES

FIG. 1 illustrates an exemplary ventricular assist device (VAD), according to an example embodiment of the present invention;

FIG. 2 illustrates an exemplary VAD system implanted in a human body, according to an example embodiment of the present invention;

FIG. 3 illustrates an artificial heart valve, according to an example embodiment of the present invention; and

FIG. 4 Illustrates an exemplary implanted coronary stent, according to an example embodiment of the present invention.

### DETAILED DESCRIPTION OF THE INVENTION

Tecarfarin is a novel vitamin K antagonist that is structurally similar to warfarin, but with several distinguishing features that potentially allow it to provide more consistent anticoagulation therapy in circumstances where warfarin is used but may be less reliable. The major difference between tecarfarin and warfarin, as a consequence of its unique structure, lies in its metabolism. Unlike warfarin, tecarfarin's metabolism does not proceed via the cytochrome P450 system; instead, it involves hydrolysis via by human carboxylesterase 2 (hCE-2), which is widely distributed throughout the body. This avoidance of cytochrome P450 substantially reduces tecarfarin's potential for drug-drug interactions and the additional risk that cytochrome P450 genetic variants can affect active drug levels.

Tecarfarin, being a vitamin K antagonist like warfarin but with improved pharmacological properties, offers a more suitable alternative for patients with implanted cardiac devices who require chronic anticoagulation, potentially combining the benefits of established efficacy with enhanced safety and ease of management. Implantation is the process or act of inserting or grafting a medical device (e.g., mechanical circulatory support device), tissue, organ, or any other biological or man-made material into the body, or onto the surface of a body tissue or organ. This process can typically be performed through surgical, minimally invasive, or non-surgical methods, depending on the nature of the material or device being implanted and the intended site of implantation

A blood pump is a mechanical device designed to circulate blood through the circulatory system or bypass sections of it, for the purpose of supporting or temporarily substituting the heart's function. This includes devices used in cardiac surgery, such as heart-lung machines, as well as VADs, LVADs and total artificial hearts (TAH) for patients with heart failure. Blood pumps can be external or implantable, with the potential for implantation within the thorax among other locations. They may operate continuously or in pulsatile mode to mimic the heart's natural rhythm.

Ventricular assist devices (VADs) are sophisticated mechanical pumps designed to support heart function and blood flow in patients with severe heart failure or weakened hearts. These devices can be used temporarily or permanently to supplement or replace the pumping action of the heart's ventricles, helping to maintain organ perfusion and stabilize hemodynamics. VADs consist of several components, including an implantable pump connected to the heart via tubes, an external controller, and a power source. They can be implanted in various configurations depending on the specific needs of the patient and may support either the right, left, or both ventricles. VADs serve multiple purposes, acting as a bridge to transplantation for patients awaiting a donor heart, a bridge to recovery for those whose hearts may regain function, or as destination therapy for individuals ineligible for transplantation. These devices have significantly improved survival rates and quality of life for patients with advanced heart failure, offering them increased mobility and the ability to return to many daily activities.

A Left Ventricular Assist Device (LVAD) is a mechanical pump implanted in patients with advanced heart failure to support the function of the left ventricle, the main pumping chamber of the heart. The LVAD helps circulate blood from the left ventricle to the aorta, effectively assisting the weakened heart in maintaining adequate blood flow to the rest of the body. LVADs benefit patients with end-stage heart failure who have exhausted other treatment options and experience symptoms even at rest. Example commercially available LVADs include the HeartMate 3, produced by Abbott Laboratories; the BrioVad, produced by BrioHealth Solution Inc.; the Evaheart 2, produced by EvaHeart, Inc.; and the HVAD, formerly manufactured by Medtronic. The LVAD system typically consists of an internal pump connected to the heart, an external controller, and a power source. It works by continuously drawing blood from the left ventricle and pumping it into the aorta, thereby reducing the workload on the native heart and improving organ perfusion.

The LVAD can be implanted in the patient's thorax below the heart with one end inserted into the apex of the left ventricle and the other inserted into the ascending aorta. The LVAD can divert blood from the weakened left ventricle and pumps it to the aorta; whereas sometimes the LVAD operates in parallel with the heart, such that either can supply blood to the aorta. The LVAD is typically intended for long-term use, in which encompasses the period of time the LVAD is implanted in the thorax, the time in which LVAD retains battery power, or the combination thereof.

A total artificial heart (TAH) is a mechanical device that replaces both the left and right ventricles of the heart, the chambers that pump blood throughout the body. TAHs can be used as temporary while patients wait for a heart transplant, but can also be used as a permanent solution for patients who are not eligible for a transplant. The SynCardia Total Artificial Heart by SynCardia Systems is an example of a commercially available TAH.

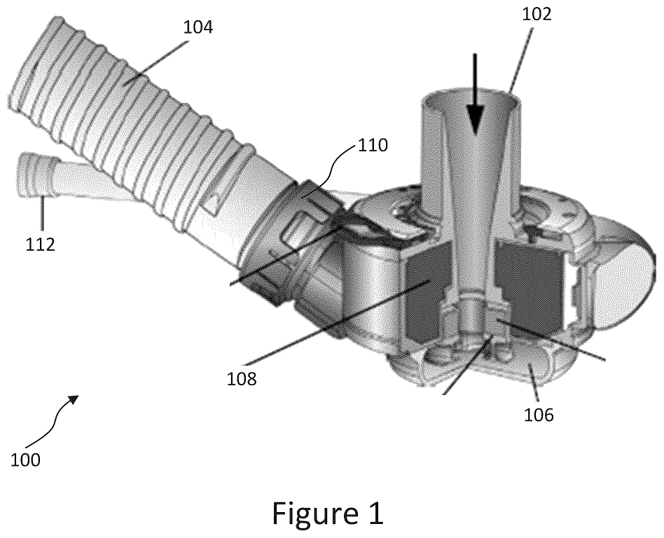

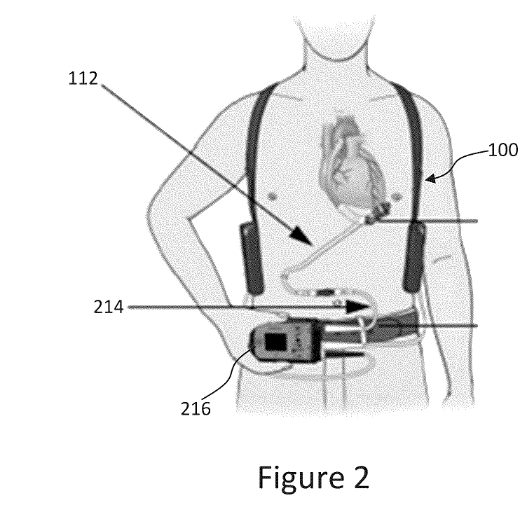

Referring to FIG. 1, an example heart pump 100 is illustrated. The heart pump includes an inflow cannula 102 and an outflow graft 104. Preferably the outflow graft 104 includes a bend relief. Blood flows in through the inflow cannula 102 into a pump chamber 106 and out the outflow graft 104. A motor 108 causes the blood to flow from the inflow cannula 102 to the outflow graft 104. The outflow graft 104 is attached via a slide lock 110. A driveline 112 provides power to the pump 100 and data communication to and from the heart pump 100. Referring to FIG. 2, the heart pump 100 of FIG. 1 is illustrated implanted into a human body. In this Figure, the driveline 112 is connected to a module cable 214 for power and data communications with a controller 216 that controls the heart pump 100.

Valvular heart disease is a disorder affecting one or more of the heart's valves including the mitral, aortic, tricuspid, and pulmonary valves, which regulate blood flow through the heart's chambers and the body. A patient may be afflicted with valve stenosis, the narrowing and restriction of blood flow; valve regurgitation, the leaking of blood back through the valve; atresia, a congenital or acquired condition in which the heart valve has no opening at all; or any combination thereof.

The primary function of heart valves is to allow unidirectional blood flow while preventing backflow, maintaining proper cardiac function. Prosthetic heart valves, including mechanical, bioprosthetic or tissue valves are designed to replace damaged or diseased natural heart valves. Mechanical valves are typically made from durable materials like pyrolytic carbon, titanium, and medical-grade fabric, ensuring longevity and functionality. Bioprosthetic and tissue valves are either in whole or in part live bio tissues. Prosthetic heart valves work by mimicking the action of natural heart valves, opening and closing with each heartbeat to regulate blood flow through the heart chambers.

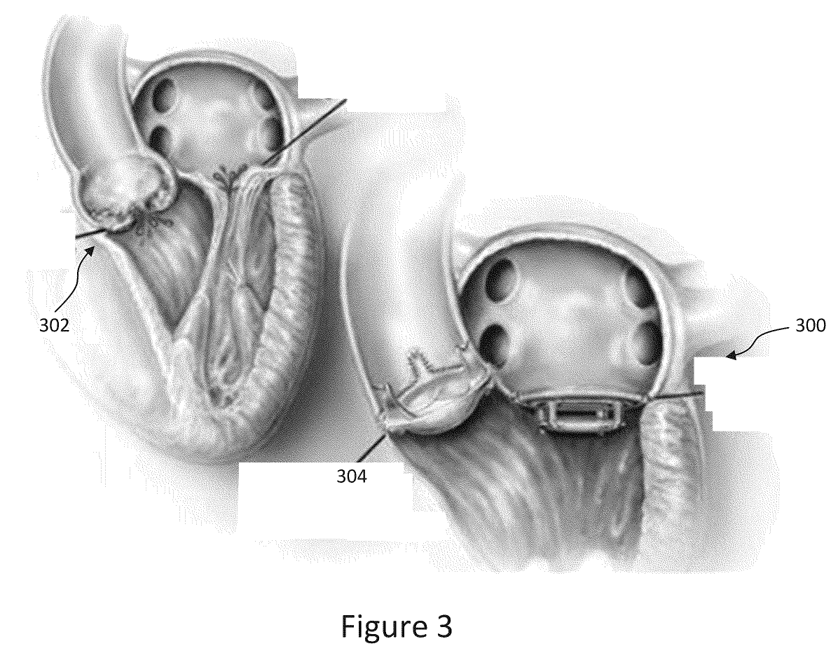

Examples of commercially available mechanical heart valves include the Masters Series and Regent™ valves from Abbott (which feature a bi-leaflet design with an 85-degree opening angle for optimal hemodynamics); Perimount and Sapien valves by Edwards Lifescience; Surgical bioprosethetic valves and TAVR systems by Medtronic and others. These valves benefit patients requiring long-term valve replacement, particularly younger individuals who can tolerate lifelong anticoagulation therapy. The manufacturing process involves precision engineering to create leaflets, orifice rings, and sewing cuffs that withstand the demanding cardiac environment. Referring to FIG. 3, a heart valve 300 is illustrated. On the left side is illustrated a diseased heart valve 302. On the right side is an illustration of a valve replacement 304.

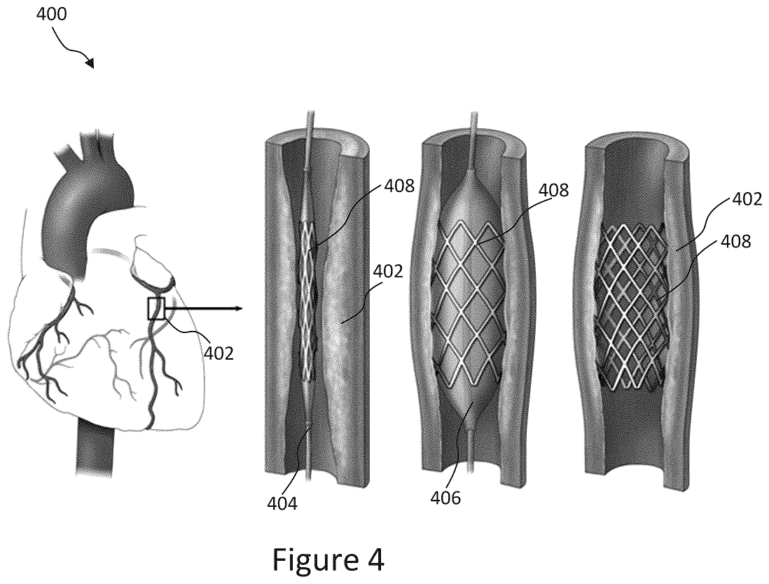

Coronary stenting is a minimally invasive procedure used to treat narrowed or blocked coronary arteries. The stent is typically made of a metal mesh tube and is deployed into the artery to keep it open. Referring to FIG. 4, coronary stenting 400 is illustrated. The involved vessel 402 is depicted on the left; moving left to right, the delivery catheter 404 is positioned at the site of the obstructing lesion in the coronary artery 402. A ballon 406 is expanded to deliver the stent 408 into the coronary artery 402; and—at far right—the delivery catheter 404 has been withdrawn leaving the implanted expanded coronary stent 408 in place and permitting normal blood flow across the previously obstructed area of the vessel 402.

All of the above conditions require treatment with an anticoagulant to reduce the likelihood of thrombus formation. In the present disclosure, the specific new anticoagulant employed is Tecarfarin, which refers to a compound with the chemical name 1,1,1,3,3,3-hexafluoro-2-methyl-2-propanyl 4-[(4-hydroxy-2-oxo-2H-chromen-3-yl)methyl] benzoate. Tecarfarin has the molecular formula C₂₁H₁₄F₆O₅ and molar mass 460.328 g·mol⁻¹. The structural formula is shown below.

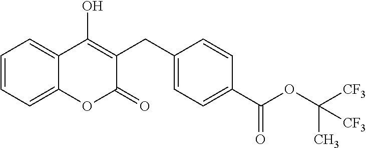 [SMILES: CC(OC(=O)c1ccc(Cc2c(O)c3ccccc3oc2=O)cc1)(C(F)(F)F)C(F)(F)F]

Tecarfarin is a vitamin K antagonist and exerts its anticoagulant effect by interfering with the vitamin K cycle in the liver, which is essential for the synthesis of several clotting factors. Specifically, tecarfarin inhibits the vitamin K epoxide reductase (VKORC1) enzyme, which is responsible for converting vitamin K epoxide back to its active reduced form, vitamin K hydroquinone. This reduced form of vitamin K is a cofactor required for the gamma-carboxylation of glutamate residues on clotting factors II, VII, IX, and X, as well as anticoagulant proteins C and S. By blocking the regeneration of active vitamin K, tecarfarin reduces the carboxylation of these clotting factors, leading to the production of non-functional or less active clotting factors that cannot effectively participate in the coagulation cascade. As a result, the overall clotting ability of the blood is diminished, thereby preventing or reducing the risk of thromboembolic events. This mechanism of action is well-established for vitamin K antagonists like warfarin, and tecarfarin is expected to function similarly, although its differentiated metabolism not involving the cytochrome P450 system is intended to provide a more consistent and predictable anticoagulant effect. Tecarfarin can exist in the unionized form (e.g., tecarfarin), or as a pharmaceutically acceptable salt (e.g., tecarfarin sodium).

Tecarfarin has the potential to solve challenges arising from warfarin metabolism, improve the reliability and consistency of the degree of coagulation achieved and thereby reduce the risk of both thrombotic and hemorrhagic complications. Tecarfarin is not metabolized via the cytochrome P450 system, but rather via an alternate pathway and enzyme (h-CE2) that is abundant throughout the body and essentially insaturable. Tecarfarin has a reliable and stable PK profile and PD response (anticoagulation), a well-understood mechanism of action (similar to other VKAs), with supporting clinical data. Tecarfarin provides stable anticoagulation and a high degree of time-within-therapeutic range (TTR>72%), which could be a significant advantage in preventing thromboembolic events in circumstances where warfarin is less reliable (TTR<50%).

Tecarfarin could be of particular benefit in certain patients who face challenges with traditional warfarin therapy. This includes individuals with genetic polymorphisms affecting CYP2C9, the enzyme primarily responsible for warfarin metabolism, leading to unpredictable drug responses and difficulty achieving stable anticoagulation. Additionally, patients taking concomitant medications that interact with CYP2C9, such as certain antifungals, antibiotics, or antiarrhythmics, would also likely benefit from tecarfarin's reduced potential for drug-drug interactions.

In a study in healthy volunteers evaluating the pharmacokinetics of combining tecarfarin and fluconazole-a potent CYP2C9 inhibitor-co-administration of fluconazole significantly prolonged the t½ and drug exposure to both R- and S-warfarin, while the PK of tecarfarin was unaffected by fluconazole co-administration, highlighting tecarfarin's potential for more predictable anticoagulation, especially in patients taking multiple medications.

Patients with chronic kidney disease (CKD), including those with end-stage kidney disease (ESKD), may also be better candidates for tecarfarin, as its pharmacokinetics remain relatively unchanged in the setting of impaired renal function, unlike warfarin, whose metabolism can be significantly affected. Patients with a history of unstable INR control on warfarin, despite good adherence to monitoring and dietary guidelines, could also be considered for tecarfarin to improve the stability and predictability of their anticoagulation.

The hCE-2 metabolism of tecarfarin has also been shown to be particularly beneficial in patients with chronic kidney disease (CKD), especially when coupled with genetic variations affecting CYP2C9 enzymes, as shown in a PK study of warfarin and tecarfarin in subjects with and without CKD. Mean plasma concentrations of(S)-warfarin and (R,S)-warfarin were significantly higher in the subjects with CKD than in the healthy subjects, while for tecarfarin, drug levels were not significantly higher in CKD subjects. Elimination half-life (t_{1/2}) increased for (S)-warfarin and (R,S)-warfarin and decreased for tecarfarin. Importantly, CKD significantly increased the effect of CYP2C9 genetic variation on(S)-warfarin and (R,S)-warfarin exposure, while tecarfarin exposure was similar between healthy subjects and CKD subjects regardless of CYP2C9 genotype.

Another condition where conventionally metabolized warfarin may be problematic is in patients with significant hepatic dysfunction and a need for chronic anticoagulation, especially those in whom there may be an underlying coagulopathy as a consequence of hepatic failure. Substantial variations in warfarin metabolism (which takes place in the liver) can result in wide variations in the degree of anticoagulation, and markedly higher bleeding risk. This variability in metabolism can be avoided since h-CE2 is more ubiquitously distributed throughout the body.

In a large Phase 2/3 randomized, double-blind, parallel group, active-control study comparing the degree of anticoagulation with tecarfarin versus warfarin in a general population of patients requiring chronic oral anticoagulation, the percent of time-within-therapeutic-range (TTR)—the primary efficacy endpoint—did not differ significantly between blinded warfarin and blinded tecarfarin treatment. Thus, tecarfarin was able to achieve consistency of anticoagulation in the broader population requiring chronic oral anticoagulation comparable to that of well-managed warfarin.

Importantly, in subjects who required the concomitant administration of a drug known to inhibit CYP2C9 (˜30% of the overall population) there was a significant difference in observed TTR between tecarfarin and warfarin. Furthermore, among subjects with the CYP2C9 wild type genotype there was significant improvement in TTR. In the small number of subjects concomitantly on CYP2C9 inhibitors analyzed according to CYP2C9 genotype, genotypic variants on CYP2C9 inhibitors had a higher TTR on tecarfarin compared to warfarin.

Incorporating tecarfarin into the anticoagulation regimen for patients who require chronic oral anticoagulation following certain procedures, such as cardiac device implantation, offers the potential to significantly improve their quality of life by alleviating some of the burdens associated with traditional warfarin therapy. Tecarfarin's predictable pharmacokinetic profile and reduced susceptibility to drug interactions could translate to the need for less frequent INR monitoring, as the need for constant dosage adjustments due to external factors is minimized. The convenience of less frequent monitoring can free patients from the anxiety and logistical challenges of regular blood tests, allowing them to lead more normal lives with fewer disruptions. By reducing the burden of frequent monitoring, tecarfarin can potentially enhance the quality of life for individuals. This improved quality of life can lead to better adherence to the anticoagulation regimen and ultimately improve patient outcomes.

The combination of anticoagulants and antiplatelet agents, such as tecarfarin and aspirin, is another complex area in cardiovascular therapeutics. While combining these therapies (as in patients already on chronic oral anticoagulant therapy undergoing coronary stenting and subsequent obligatory addition of dual antiplatelet therapy) may offer enhanced protection against thrombotic events in certain high-risk patients, it also carries an increased risk of bleeding complications. because antiplatelet agents (like aspirin, clopidogrel, prasugrel and ticagrelor) prevent platelet aggregation and themselves can limit the progression of thrombosis.

Following coronary stenting it is customary to treat with two complementary antiplatelet agents to maximally inhibit platelet aggregation. Combining antithrombotic agents and antiplatelet agents may provide more comprehensive protection against both fibrin-rich and platelet-rich thrombus formation in patients undergoing coronary stent implantation who are already on an oral anticoagulant to which more intense antiplatelet therapy must be added, but these patients are also at substantially higher risk of bleeding complications, and more precise control is highly desirable.

In these situations, the potential benefits of combination therapy must be carefully weighed against the increased risk of bleeding. Studies have shown that combining anticoagulants with aspirin significantly increases the risk of major bleeding events compared to anticoagulant monotherapy. For instance, in patients with atrial fibrillation already on warfarin, the addition of aspirin alone to warfarin was associated with an incremental rate of major bleeding of 1.6% per year without a significant reduction in stroke, systemic embolism, or myocardial infarction. To manage these risks, clinicians must carefully assess each patient's individual risk profile, taking into account factors such as age, renal function, history of bleeding, and concomitant medications. In these circumstances tecarfarin may offer some advantages in combination therapy due to its unique metabolism via carboxyl esterase rather than the cytochrome P450 system, potentially reducing drug interactions and providing more stable anticoagulation, and less risk of concomitant bleeding.

Prothrombin Time/International Normalized Ratio (PT/INR) testing plays a central role in managing anticoagulation therapy, particularly with vitamin K antagonists like warfarin and tecarfarin. The PT/INR test measures the time it takes for blood to clot and provides a standardized ratio to assess the effectiveness of anticoagulation, with the target range typically set between 2.0 and 3.0 for most indications, including blood pumps, mechanical heart valves coronary stenting. In practice, PT/INR testing is typically initiated shortly after starting anticoagulant therapy, with more frequent testing (e.g., daily or every other day) during the initial stabilization phase to determine the appropriate maintenance dosage.

Once the INR is within the target range, the frequency of testing can be reduced to weekly or bi-weekly intervals, depending on the stability of the INR and individual patient factors. Dosage adjustments are made based on the INR results, with incremental increases or decreases in tecarfarin dosage to bring the INR back within the therapeutic range, guided by established algorithms and clinical judgment. Patients may also use home monitoring INR devices for more convenient testing, but it is essential to have regular follow-up with a healthcare provider to review results and make necessary adjustments. This rigorous monitoring and adjustment process is essential to maintain safe and effective anticoagulation, minimizing the risk of both thromboembolic and bleeding complications.

International normalized ratio or “INR” refers to a critical measure for monitoring the clotting tendency of blood, particularly in patients undergoing anticoagulant therapy with Vitamin K antagonists such as Tecarfarin. Measuring the degree of anticoagulation is particularly important for monitoring and adjusting medication dosages to achieve optimal therapeutic effects, ensuring patient safety and effective management of coagulation. The term “PT/INR test” refers to a prothrombin time (PT) test that is a diagnostic evaluation used to measure the time it takes for blood to clot, expressed through the international normalized ratio (INR). A PT/INR test can be carried out after the administration of Tecarfarin to achieve and maintain an INR of, e.g., 2-3. A PT/INR test may be conducted in a clinical setting or by a patient with the assistance of a home monitoring INR medical device.

The specific INR ranges and dosages for warfarin and tecarfarin are carefully determined to balance the risks of thrombosis and bleeding. For most patients, the target INR range for warfarin therapy is 2.0-3.0, with a specific target of 2.5. This range is considered optimal because it provides effective anticoagulation while minimizing the risk of adverse events. INR values below 2.0 are associated with an increased risk of thrombosis, while values above 3.0 significantly raise the risk of bleeding complications. The narrow therapeutic window necessitates careful monitoring and dose adjustments to maintain the INR within this range.

For certain conditions, such as patients with prosthetic heart valves or refractory hypercoagulable states, a higher target INR range of 2.5-3.5 may be recommended. This higher range provides more intense anticoagulation for patients at greater risk of thrombotic events, though it also carries an increased risk of bleeding. The specific target INR is individualized based on the patient's condition and risk factors.

Regarding dosage, there is no standard dose of a Vitamin K antagonist that applies to all patients. The optimal maintenance dose of warfarin varies widely from person to person and can range from 0.5 mg to 20 mg or more daily. This variability is due to individual differences in metabolism, diet, concurrent medications, and genetic factors that influence sensitivity. The goal is to administer the lowest effective dose that maintains the target INR. Dosage adjustments are typically made by changing the total weekly warfarin dose, often in increments of 5-20% based on the current INR and the magnitude of deviation from the target range.

Regular monitoring is paramount when managing anticoagulation with tecarfarin, particularly in the initial stages of treatment following implantation of a cardiac device. Due to the complexity of achieving stable anticoagulation in this patient population, frequent INR checks are essential, ideally daily or every other day, until the INR has stabilized within the desired therapeutic range, which is typically between 2.0 and 3.0, though this may vary based on individual patient factors and the type of device implanted. Once the INR is stable, the frequency of monitoring can be reduced to weekly or bi-weekly intervals, but vigilance remains crucial. Beyond INR, it's important to monitor other relevant parameters. Renal function should be assessed periodically, as kidney impairment can affect drug clearance and increase the risk of bleeding. Patients should also be educated about the signs and symptoms of both bleeding (e.g., unusual bruising, nosebleeds, blood in urine or stool) and thrombosis (e.g., chest pain, shortness of breath, swelling in extremities), and instructed to report any concerns promptly. Regular clinical assessments, including physical examinations and review of concomitant medications, are also essential to optimize tecarfarin therapy and minimize potential complications.

The incorporation of home monitoring INR devices into the management of anticoagulation with tecarfarin offers several advantages for patients on chronic oral antithrombotic therapy. Home monitoring empowers patients to take a more active role in their care by allowing them to check their INR at their convenience, without the need for frequent clinic visits. This can improve adherence to monitoring schedules and provide more timely feedback on the effectiveness of their anticoagulation regimen. By detecting out-of-range INR values promptly, patients and their healthcare providers can make timely adjustments to the tecarfarin dosage, potentially reducing the risk of both thromboembolic and bleeding complications. However, home monitoring also has limitations. The accuracy of home INR devices can vary, and it is essential to ensure that patients receive proper training on how to use the device correctly and interpret the results. Furthermore, home monitoring should not replace regular follow-up with a healthcare provider. Patients should still have periodic clinic visits for comprehensive assessments, including physical examinations, review of concomitant medications, and confirmation of home INR results. In integrating home monitoring into the overall management plan, clear communication channels are essential. Patients should know how to contact their healthcare provider with any concerns or questions, and providers should have systems in place to review home INR results promptly and provide appropriate guidance.

Before the present invention, tecarfarin was not used in all of the circumstances described herein. Tecarfarin is an experimental drug and not currently approved anywhere in the world for any indications, and has not been evaluated for uses with blood pumps or coronary stenting; existing data are from a relatively small, more general population of patients requiring anticoagulation (including some with prosthetic heart valves) or as used in healthy volunteers. There may be other complex unanticipated issues in critically ill patients that can affect safety and efficacy of antithrombotic therapy. Tecarfarin has a different structure and different metabolism from other VKA anticoagulants, and other similar coumarin derivatives have varying degrees of antithrombotic efficacy. As with any novel therapy such as those disclosed herein, further research and trials are necessary to fully evaluate the safety and efficacy of tecarfarin in combination with antiplatelet agents.

The following is a general guideline for initiating tecarfarin therapy in lieu of warfarin. Actual clinical practice and recommendations may vary, and a qualified healthcare professional should always guide treatment decisions.

In one example, initiating tecarfarin therapy can begin with a starting dose of 10 mg to 20 mg once daily, though this may be adjusted based on individual patient characteristics and clinical circumstances. Factors influencing the optimal tecarfarin dosage include age, weight, renal function, concomitant medications, and the specific indication for anticoagulation. Elderly patients or those with impaired renal function may require lower initial doses to minimize the risk of bleeding complications. Close monitoring of the International Normalized Ratio (INR) is crucial during the initiation phase, with daily or every-other-day testing recommended until the INR stabilizes within the target range of 2.0 to 3.0 for most patients, but may be 2.5-3.5 for patients with increased risk of thromboembolic events, such as those with a mechanical heart valve. Subsequent dosage adjustments should be guided by INR values, typically in small increments (e.g., 2.5 mg to 5 mg) to achieve and maintain the desired therapeutic effect. While a direct transition from warfarin to tecarfarin has not been explicitly defined, a potential approach would involve overlapping the two medications for several days while closely monitoring the INR and gradually tapering the warfarin dose as the tecarfarin effect becomes evident. In patients with implanted VADs, close monitoring of the blood pump's flow rate may also be important.

### Specific Ranges, Values, and Embodiments

The specific embodiments describing the ranges and values provided below are for illustration purposes only, and do not otherwise limit the scope of the disclosed subject matter, as defined by the claims.

In specific embodiments, the Tecarfarin is administered in an amount sufficient to achieve an international normalized ratio (INR) of below 4.

In specific embodiments, the Tecarfarin is administered in an amount sufficient to achieve an international normalized ratio (INR) of above 1.

In specific embodiments, the Tecarfarin is administered in an amount sufficient to achieve an international normalized ratio (INR) of 1.5-3.5.

In specific embodiments, the Tecarfarin is administered in an amount sufficient to achieve an international normalized ratio (INR) of 1.75-3.25.

In specific embodiments, the Tecarfarin is administered in an amount sufficient to achieve an international normalized ratio (INR) of 1.5-2.5.

In specific embodiments, the Tecarfarin is administered in an amount sufficient to achieve an international normalized ratio (INR) of 2-3.

In specific embodiments, the Tecarfarin is initially administered to the patient 2-6 days after the implantation.

In specific embodiments, the Tecarfarin is initially administered to the patient 3-5 days after the implantation.

In specific embodiments, the Tecarfarin is co-administered with 50-400 mg aspirin once daily.

In specific embodiments, the Tecarfarin is co-administered with 75-325 mg aspirin once daily.

In specific embodiments, the Tecarfarin is co-administered with 100-200 mg aspirin once daily.

In specific embodiments, the Tecarfarin is co-administered without aspirin or other antiplatelet agents.

In specific embodiments the Tecarfarin is administered with clopidogrel, prasugrel, ticagrelor, dipyridamole, persantine, or low molecular-weight dextran.

In specific embodiments the tecarfarin is administered with multiple antiplatelet agents.

In specific embodiments, up to 40 mg a day of Tecarfarin is administered to the patient.

In specific embodiments, up to 30 mg a day of Tecarfarin is administered to the patient.

In specific embodiments, 10 mg, 20 mg, or 30 mg a day of Tecarfarin is administered to the patient.

In specific embodiments, after an initial administration of Tecarfarin, a PT/INR test is carried out on the patient, and a subsequent administration of Tecarfarin is carried out, wherein the subsequent administration of tecarfarin is at a higher dose compared to the initial administration of Tecarfarin.

In specific embodiments, after an initial administration of Tecarfarin, a PT/INR test is carried out on the patient, and a subsequent administration of tecarfarin is carried out, wherein the subsequent administration of tecarfarin is at a lower dose compared to the initial administration of tecarfarin.

In specific embodiments, after an initial administration of tecarfarin, a PT/INR test is carried out on the patient, and a subsequent administration of tecarfarin is carried out, wherein the subsequent administration of tecarfarin is at the same dose as the initial administration of tecarfarin.

In specific embodiments, after an initial administration of tecarfarin, a PT/INR test is carried out on the patient, and a subsequent administration of tecarfarin is carried out, and the process is repeated one or more times sufficient to maintain the international normalized ratio (INR) values between 1.5-3.5.

In specific embodiments, after an initial administration of tecarfarin, a PT/INR test is carried out on the patient, and a subsequent administration of tecarfarin is carried out, and the process is repeated one or more times sufficient to maintain the international normalized ratio (INR) values between 1.5-3.25.

In specific embodiments, after an initial administration of tecarfarin, a PT/INR test is carried out on the patient, and a subsequent administration of tecarfarin is carried out, and the process is repeated one or more times sufficient to maintain the international normalized ratio (INR) values between 1.5-3.

In specific embodiments, after an initial administration of tecarfarin, a PT/INR test is carried out on the patient, and a subsequent administration of tecarfarin is carried out, and the process is repeated one or more times sufficient to maintain the international normalized ratio (INR) values between 1.5-2.5.

In specific embodiments, after an initial administration of tecarfarin, a PT/INR test is carried out on the patient, and a subsequent administration of tecarfarin is carried out, and the process is repeated one or more times sufficient to maintain the international normalized ratio (INR) values between 2-3.

In specific embodiments, adjusting a dosage of tecarfarin in response to receiving information from the PT/INR test via a home monitoring INR device indicating an INR value outside a threshold of between 1.5-3.5.

In specific embodiments, adjusting a dosage of tecarfarin in response to receiving information from the PT/INR test via a home monitoring INR device indicating an INR value outside a threshold of between 1.5-3.

In specific embodiments, adjusting a dosage of tecarfarin in response to receiving information from the PT/INR test via a home monitoring INR device indicating an INR value outside a threshold of between 1.5-2.5.

In specific embodiments, adjusting a dosage of tecarfarin in response to receiving information from the PT/INR test via a home monitoring INR device indicating an INR value outside a threshold of between 2-3.

In specific embodiments, adjusting a dosage of tecarfarin in response to receiving information from the blood pump via a human machine interface indicating that a flow rate of blood is sustained below a threshold of 4.0 liters per minute (lpm).

In specific embodiments, adjusting a dosage of tecarfarin in response to receiving information from the blood pump via a human machine interface indicating that a flow rate of blood is sustained below a threshold of 3.5 liters per minute (lpm).

In specific embodiments, adjusting a dosage of tecarfarin in response to receiving information from the blood pump via a human machine interface indicating that a flow rate of blood is sustained below a threshold of 3.0 liters per minute (lpm).

In specific embodiments, adjusting a dosage of tecarfarin in response to receiving information from the blood pump via a human machine interface indicating that a flow rate of blood is sustained below a threshold of 2.5 liters per minute (lpm).

In specific embodiments, the tecarfarin is administered in a single daily dose.

In specific embodiments, the tecarfarin is administered in divided doses, twice daily.

In specific embodiments, the tecarfarin is administered orally.

In specific embodiments, the tecarfarin is administered intravenously.

In specific embodiments, the tecarfarin is administered via a nasogastric tube.

In specific embodiments, the tecarfarin is formulated as a tablet.

In specific embodiments, the tecarfarin is formulated as a capsule.

In specific embodiments, the tecarfarin is formulated as an oral suspension.

In specific embodiments, the tecarfarin is administered at approximately the same time each day.

In specific embodiments, the PT/INR test is performed using a point-of-care testing device.

In specific embodiments, the PT/INR test is performed in a laboratory setting.

In specific embodiments, the patient self-manages their tecarfarin dosage based on home INR monitoring, with physician oversight.

In specific embodiments, a healthcare provider adjusts the tecarfarin dosage based on PT/INR results.

In specific embodiments, the flow rate of the blood pump is monitored continuously.

In specific embodiments, the flow rate of the blood pump is monitored intermittently.

In specific embodiments, a decrease in blood pump flow rate is defined as a reduction of at least 0.5 LPM from baseline.

In specific embodiments, a decrease in blood pump flow rate is defined as a reduction of at least 1.0 LPM from baseline.

In specific embodiments, the tecarfarin is administered to a patient with a CYP2C9 polymorphism.

In specific embodiments, the CYP2C9 polymorphism is CYP2C9*2 or CYP2C9*3.

In specific embodiments, the tecarfarin is administered to a patient taking a drug that is a CYP2C9 inhibitor.

In specific embodiments, the tecarfarin is administered to a patient taking a drug that is a CYP2C9 inducer.

In specific embodiments, the tecarfarin is administered to a patient taking a drug that is a CYP2C9 substrate.

In specific embodiments, the CYP2C9 interacting drug is fluconazole, amiodarone, or cimetidine.

In specific embodiments the tecarfarin is administered to a patient with impaired renal function.

In specific embodiments, the tecarfarin is administered to a patient with chronic kidney disease (CKD).

In specific embodiments, the chronic kidney disease is end-stage kidney disease (ESKD).

In specific embodiments the tecarfarin is administered to a patient on dialysis.

In specific embodiments, the tecarfarin is administered to a patient with a history of unstable INR control on warfarin.

In specific embodiments, “unstable INR control” is defined as spending more than 40% of the time outside the target INR range on warfarin.

In specific embodiments, the tecarfarin is administered to a patient who has experienced a bleeding event while on warfarin.

In specific embodiments, the tecarfarin is administered to a patient who has experienced a thromboembolic event while on warfarin.

In specific embodiments the tecarfarin is administered to a patient who has recently undergone coronary stent placement and requires chronic antithrombotic therapy.

In specific embodiments the tecarfarin is administered to a patient with hepatic failure.

In specific embodiments the tecarfarin is administered to a patient with hepatic failure and abnormal coagulation parameters.

1. A method of inhibiting coagulation in a patient having undergone implantation of a cardiac device, the method comprising administering tecarfarin to the patient, in an amount and for a period of time, effective to inhibit coagulation.

2. The method of claim 1 wherein the cardiac device is a blood pump, prosthetic heart valve or a coronary stent.

3. The method of claim 2, where the tecarfarin is administered in an amount sufficient to achieve an international normalized ratio (INR) of 2-3.

4. The method of claim 2, where the inhibiting coagulation is intended to prevent blood clots in the circulatory system.

5. The method of claim 2, where inhibiting coagulation protects against stroke in a patient having valvular heart disease, prosthetic heart valves, or a combination thereof.

6. The method of claim 2, wherein a prosthetic heart valve is not a mechanical heart valve.

7. The method of claim 2, wherein the tecarfarin is initially administered to the patient 3-5 days after the implantation.

8. The method of claim 2, wherein the tecarfarin is co-administered with aspirin or another antiplatelet agent.

9. The method of claim 2, wherein the tecarfarin is co-administered with 75-325 mg aspirin once daily.

10. The method of claim 2, wherein the tecarfarin is administered in the absence of aspirin.

11. The method of claim 2, wherein the tecarfarin is co-administered with heparin.

12. The method of claim 11, wherein the heparin is discontinued after maintaining an acceptable international normalized ratio (INR) value of 2-3.

13. The method of claim 2, wherein the blood pump contains an inflow cannula, a pump cover, a lower housing, a screw ring attaching the pump cover to the lower housing, a motor, an outflow graft, and a pump cable.

14. The method of claim 2, wherein the blood pump is a left ventricular assist device (LVAD), intended for long-term implantation in the patient.

15. The method of claim 2, wherein the blood pump is a HeartMate3™ (Abbot, Chicago, Illinois) left ventricular assist system.

16. The method of claim 2, wherein the blood pump is a CorWave (France) left ventricular assist system.

17. The method of claim 2, wherein the blood pump is a HeartWave™ HVAD™ (Medtronic, Minneapolis, Minnesota) system.

18. The method of claim 2, wherein the blood pump is an EXCOR® Pediatric (Berlin Heart, Berlin, Germany) left ventricular assist system.

19. The method of claim 2, wherein the blood pump is a HeartAssist5® (ReliantHeart, Houston, Texas) left ventricular assist system.

20. The method of claim 2, wherein the blood pump is a Jarvik 2000® (Jarvik Heart™, New York, New York) ventricular assist system.

21. The method of claim 2, wherein the blood pump is an EVAHEART®2 (EvaHeart, Bellaire, Texas) left ventricular assist system.

22. The method of claim 2, wherein the advanced heart failure is advanced refractory left ventricular heart failure.

23. The method of claim 2, which is a method for the mitigation of thromboembolic complications in a patient diagnosed with advanced refractory left ventricular heart failure.

24. The method of claim 2, wherein up to 30 mg a day of tecarfarin is administered to the patient.

25. The method of claim 2, wherein 10 mg, 20 mg, or 30 mg of tecarfarin is administered once daily to the patient.
